(19) United States
(12) Patent Application Publication
(10) Pub. No.: US 2015/0297265 A1
(43) Pub. Date: Oct. 22, 2015

(54) Remote Operated Adjustable Spine Device

(71) Applicant: Alan J. Arena, Chino, CA (US)

(72) Inventor: Alan J. Arena, Chino, CA (US)

(21) Appl. No.: 14/625,555

(22) Filed: Feb. 18, 2015

Related U.S. Application Data
(60) Provisional application No. 61/982,661, filed on Apr. 22, 2014.

(51) Int. Cl. A61B 17/70

(52) U.S. Cl. CPC A61B 17/7016; A61B 17/7034

(57) ABSTRACT

Fusion of spinal vertebrae requires fixation across the disk. This fixation needs to be rigid to allow the bone to grow across the region being fused. The current approach fixes this section of spine during surgery. The patient is under anesthesia and lying flat as the surgeon sets the lordosis for this region of the spine. If the lordosis is not set right the adjacent vertebrae have to adapt to correct the lordosis. This causes the fused portion of the spine to have incorrect curvature and the adjacent vertebrae to carry higher loads. Correction of this fixated section would require additional surgery. This remote operated adjustable spine device allows the lordosis of the area to be fused to be adjusted after surgery with the patient awake and in a natural standing position. The spine device also incorporates benefits for spinal sclerosis stabilization using a simpler smaller implanted device.

### CROSS REFERENCE TO RELATED APPLICATIONS

This application claims the benefit of Provisional Application Ser. No. 61/982,661 filed Apr. 22, 2014 the entire contents of which is hereby expressly incorporated by reference herein.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This application relates to surgical systems, assemblies, devices, and methods that may be used for less invasive and/or minimally invasive surgery, and in particular relates to surgical systems, assemblies, devices, and methods that may relate to gaining access to and/or treatment of the spine.

2. Description of Related Art Including Information Disclosed Under 37 CFR 1.97 and 1.98.

One type of conventional treatment of spinal pathologies is spinal stabilization, also known as intervertebral stabilization. Intervertebral stabilization desirably prevents relative motion between vertebrae of the spine. By preventing movement, pain can be reduced. Stabilization can be accomplished by various methods. One method of stabilization is spinal fusion. Another method of stabilization is fixation of any number of vertebrae to stabilize and prevent movement of the vertebrae. In addition, where compression or subsidence of the disc and/or facet joints has occurred, the physician can utilize fusion devices such as pedicle screw and rods systems, or interbody fusion cages, to elevate or “jack up” the compressed level, desirably obtaining a more normal anatomical spacing between the vertebral bodies.

The invention relates to the remote operation of a device for locking and or stabilizing a linear positioning device. More particularly this invention relates to a spine locking and or stabilizing a linear positioning device that is remotely operated to allow the invention to be implanted. The force required to actuate the device is not related to its holding force. This remote operated adjustable spine device remotely unlocks and locks the position holding. Positioning is accomplished by patient motion as directed by the surgeon, physician, medical technician, or nurse.

Current art for implantable orthopedic and spinal medical devices is to implant a fixed, non-moving device that is fixed at the time of the surgery. If an adjustment to the device is required a second surgery is necessitated.

A number of patents and or publications have been made to address these issues. Exemplary examples of patents and or publication that try to address this/these problem(s) are identified and discussed below.

U.S. Pat. No. 8,672,979 issued on Mar. 18, 2014 to Randolph C. Bishop discloses a “Spinal stabilization system for the stabilization and fixation of the lumbar spine and method for using same”, is a fixed lumbar stabilization system, it does not allow for any non-invasive post-operative adjustment to the surgically set lordosis. The common factor in this, and other patents is that they make it easier for the surgeon to install the spine stabilization apparatus, but none allow for the spine stabilization apparatus to adjust the lumbar spine lordosis angle to be adjusted post-operatively with the patient in a vertical standing position; none allow for the patient to participate in setting the lordosis angle; none allow for the fine tuning of the lordosis angle; none allow for any non-invasive post-operative adjustment to the lordosis angle.

U.S. Pat. No. 8,197,490 issued on Jun. 12, 2012 to Scott Pool et al., discloses a “Non-invasive adjustable distraction system” that is an adjustable device, it requires a complex external control unit and it cannot address a single level vertebral stabilization because of its large size. This device externally forces the spine to a position. This invention incorporates all of the benefits and familiarity of the current art for spine stabilization systems while adding the ability to post-operative fine tune the lumbar spine lordosis non-invasively with the patient in a normal standing position. The Pool patent also incorporates all of the benefits and familiarity with spinal sclerosis stabilization and correction by using smaller implanted devices.

What is needed is an implant that is adjustable following the initial surgery without the requirement of an additional surgery. The disclosed device reduces medical costs (surgeon, staff, and operating room time and expenses), is more convenient for the patient, and reduces the risk of secondary opportunistic infections.

### BRIEF SUMMARY OF THE INVENTION

It is an object of the remote operated adjustable spine device to be a remote operated locking device that uses a remote controlled orthogonal lock concept whereas the locking feature application force is independent and uncoupled, except for friction forces, from the holding force. (This is analogous to the force required to turn a door handle being independent and uncoupled, except for friction forces, from the force required to break open a closed door.).

It is an object of the remote operated adjustable spine device to operate with a more than a casual magnetic source to unlock the device but not require an external magnetic device so large that the external magnetic device becomes cumbersome.

It is another object of the remote operated adjustable spine device tallow a surgeon to adjust the patient's spine with the patient in a natural vertical position. This adjustment is accomplished using a simple procedure a couple of days after the surgery is completed. The surgeon evaluates the patient's lordosis in the lumbar spine with the patient in a standing position. The surgeon then unlocks the implanted adjustable spine devices; a pair of devices are required to stabilize the vertebral region being fused. The unlocking is achieved using a small external magnetic device placed against the back region adjacent to the adjustable spine implants. The patient standing and the adjustable spine device unlocked, the surgeon positions the patient with the patient's head properly positioned over the pelvis. With the spine in the correct position, proper lordosis is attained. With the adjustable spine device temporarily unlocked, the spine rods are now allowed to move as the spine lordosis is properly set. Once the desired spine lordosis is achieved, the external magnetic device is then moved away from the region and the adjustable spine devices lock the rods in their final position.

It is still another object of the remote operated adjustable spine device to allow a surgeon to adjust the patient's spine with the patient in a natural vertical position. This adjustment is accomplished using a simple procedure a couple of days after the surgery is completed. The surgeon evaluates the patient's spinal sclerosis condition. The surgeon then unlocks the implanted adjustable spine devices; a pair of devices are required to stabilize the vertebral region being corrected for sclerosis. The unlocking is achieved using a small external magnetic device placed against the back region adjacent to the adjustable spine implants. With the adjustable spine device unlocked, the surgeon positions the patient's spine making spinal adjustments to incrementally correct the patient's spinal sclerosis condition. With the adjustable spine device temporarily unlocked, the spine rods are now allowed to move as the spine is manually adjusted. When the surgeon is satisfied with the incremental spinal adjustment, the external magnetic device is then moved away from the region and the adjustable spine devices lock the rods in position. With the rods locked in position the spine is allowed to stabilize in its new position. This procedure is repeated until a final spinal configuration is achieved.

Various objects, features, aspects, and advantages of the present invention will become more apparent from the following detailed description of preferred embodiments of the invention, along with the accompanying drawings in which like numerals represent like components.

### BRIEF DESCRIPTION OF THE SEVERAL VIEWS OF THE DRAWING(S)

FIG. 1 shows an embodiment of the remote operated adjustable spine device used as adjustable spine/vertebrae fusion rod.

FIG. 2 shows an embodiment of the remote operated adjustable spine device used as adjustable spine/vertebrae rod spanning multiple vertebrae.

FIG. 3 shows the working operation of an embodiment of the remote operated adjustable spine device.

FIG. 4 shows the working operation of an embodiment of the remote operated adjustable spine device with optional cover.

FIG. 5 is a cut-away view of an embodiment of the remote operated adjustable spine device showing an embodiment of the internal workings of the remote operated adjustable spine device.

FIG. 6 is an expanded view of the section shown in FIG. 3 showing an embodiment of the comb feature for discretely position the remote operated adjustable spine device using comb teeth and the comb.

### DETAILED DESCRIPTION OF THE INVENTION

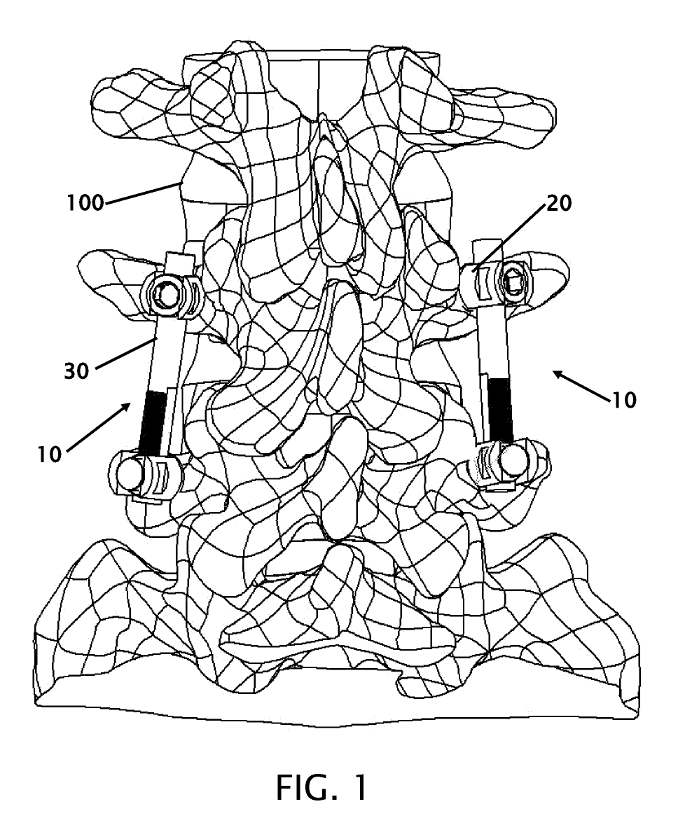

FIG. 1 shows an embodiment of the remote operated adjustable spine device used as adjustable spine/vertebrae fusion rod using a digitized representation of the lumbar spine 100 and typical pedicle screws 20 used in spinal fusions. The typical placement of one embodiment of the remote operated adjustable spine device 10 in a spinal/vertebral fusion is shown. While the remote operated adjustable spine device 10 may be shown and described spanning adjoining vertebrae, the remote operated adjustable spine device 10 can also be used across multiple vertebral fusions. For clarity in this figure, both of the optional covers have been omitted from the view.

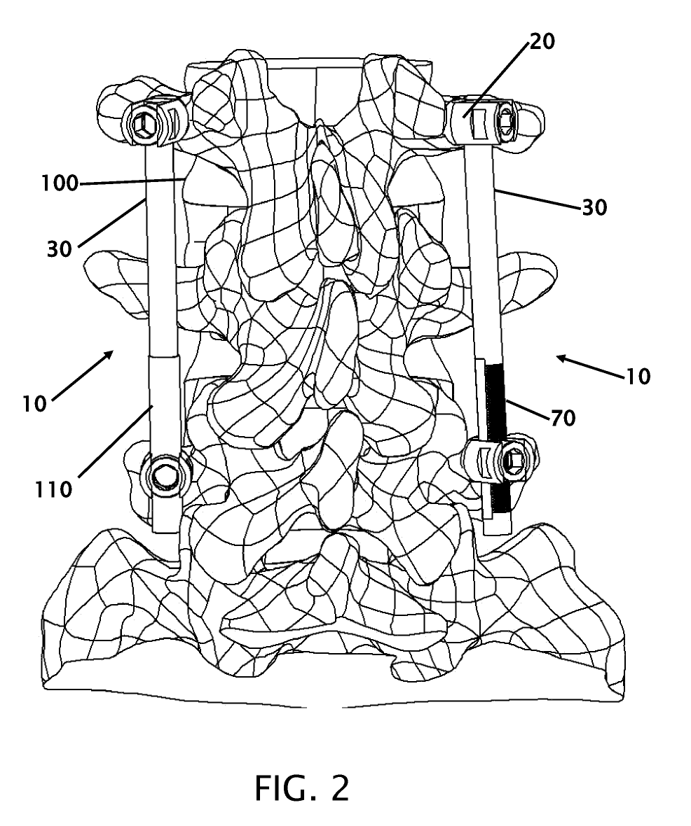

FIG. 2 shows the remote operated adjustable spine device spanning multiple vertebrae. In FIG. 2, the device is shown only spanning two vertebrae but any number of vertebrae may be spanned. The remote operated adjustable spine device 10 allows for pedicle screws 20 to be inserted across multiple vertebrae of said patient. When the remote operated adjustable spine device 10 is spans over or across more than one vertebrae it can corrects spine curvature such as spinal scoliosis in patients. The span further allows adjustable spine devices to be inserted and held in place. In this figure, one optional cover 110 is shown that covers the spline 70 on the control rod 30.

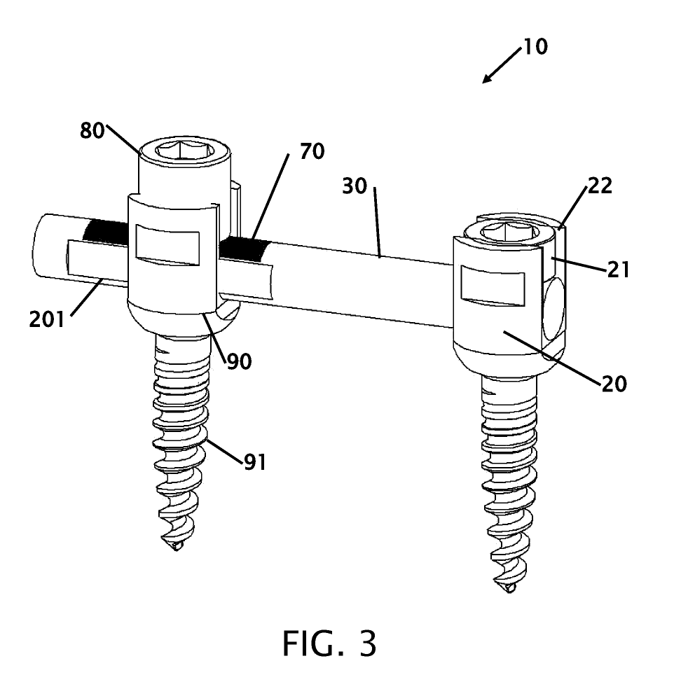

FIG. 3 shows the working operation of an embodiment of the remote operated adjustable spine device 10. In this figure, the isometric view of the embodiment of the remote operated adjustable spine device 10 is shown with a control cap 80, mated to the control tulip 90 with multi-axial pedicle screw, and a control rod 30. This figure shows an embodiment of the discrete displacement control feature 70 of the remote operated adjustable spine device 10 shows a plurality of engaging splines or comb teeth 70.

For this remote operated adjustable spine device 10 to properly function as a spinal/vertebral rod replacement, it has to remain rigid during normal operation and during the remote control adjustment operation. To do this, a pair of flats 201 are located on opposite side of the control rod 30. The pair of flats 201 prevent rotation of the control rod 30 during normal and remote control operation. The control cap 80 screws into the control tulip 90 in exactly the same manner as the nut 21 screws into the tulip 22 of a pedicle screw 20. The control tulip 90 has a positioning feature that locates the control cap 80 at the control rod 30. The positioning feature also serves to fix the control rod 30 within the depth of the control tulip 90 both during normal operation and during the remote control adjustment operation. A typical pedicle screw 20 is used to secure the free end of the control rod 30. The pedicle screw 20 fixes one end of the control rod 30 preventing translation in the x, y, and z directions and prevents rotation of the control rod 30 in pitch, roll, and yaw.

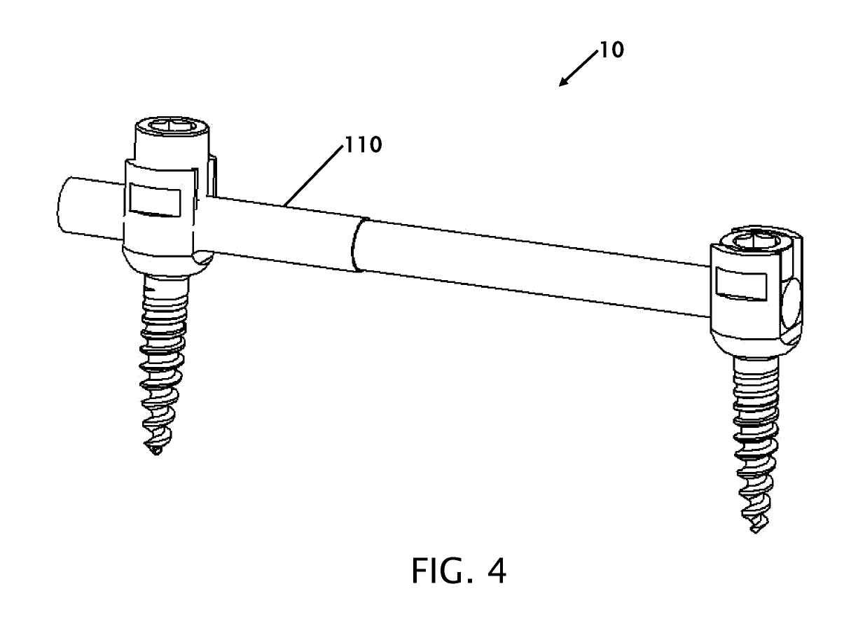

FIG. 4 shows the working operation of an embodiment of the remote operated adjustable spine device with optional cover 110. The cover 110 provided a continuous smooth surface, covering the discrete displacement control feature 70

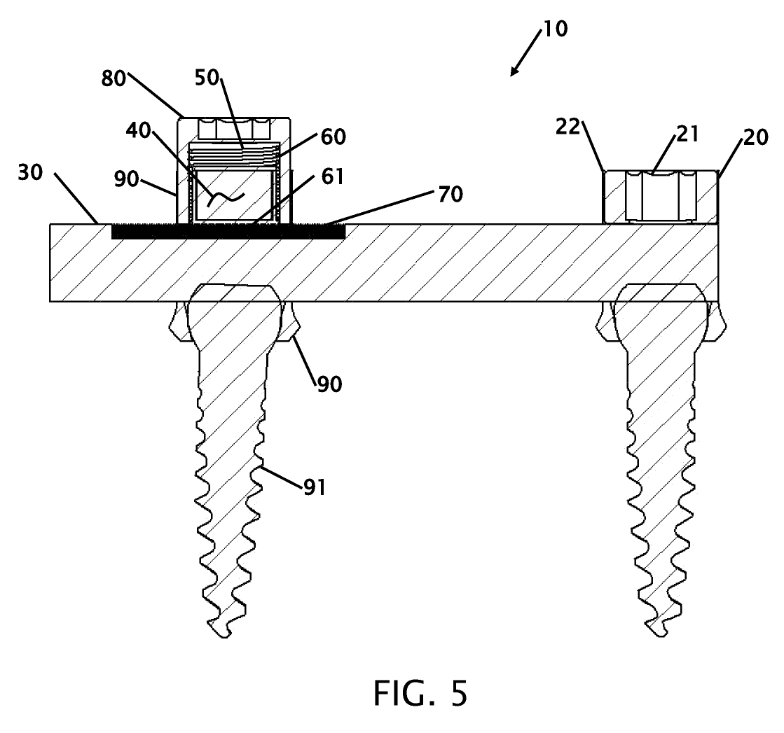

FIG. 5 is a cut-away view of an embodiment of the remote operated adjustable spine device 10 showing an embodiment of the internal workings of the remote operated adjustable spine device 10. This embodiment shows the mating comb teeth 61 that both lock and allow for the discrete positioning of the remote operated adjustable spine device when combined with the complimentary comb teeth 70 on the control rod 30. The comb teeth 61 are just one feature of the magnet housing 60, which also contains the magnet 40, centers the spring 50 and also contains features to keep the comb teeth 61 aligned with the mating comb teeth 70 of the control rod 30. The remote control is affected by applying an external magnetic field to pull the magnet 40 against the positioning spring 50 that allows the combed teeth 61 and 70 to disengage and allow the control shaft 30 to be shifted linearly.

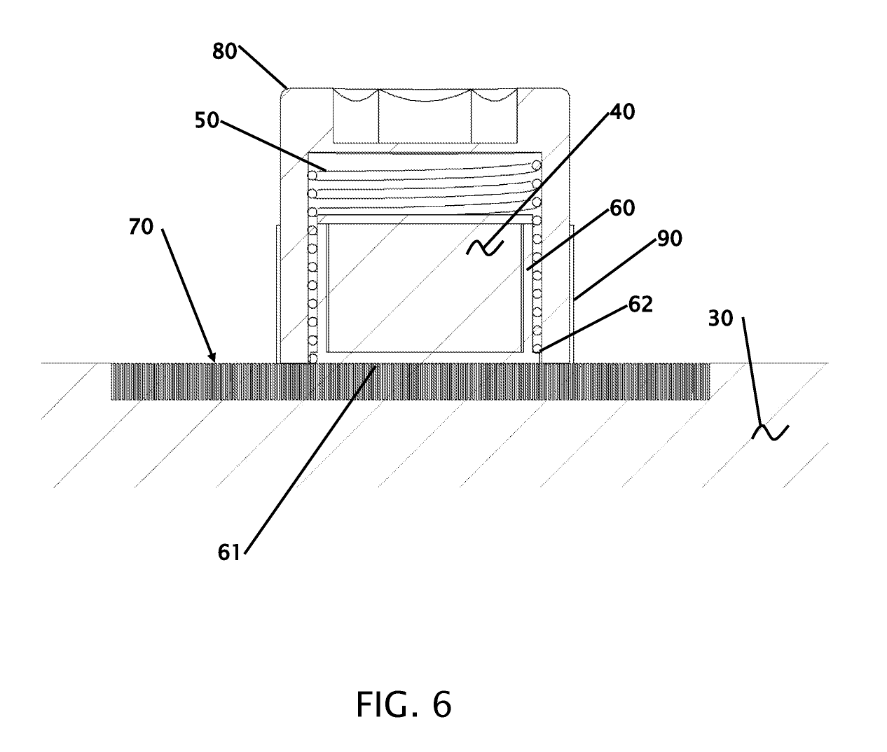

FIG. 6 is an expanded view of the section shown in FIG. 3 showing an embodiment of the comb feature for discretely position the remote operated adjustable spine device using comb teeth 61 and the comb 70. In this figure multiple features of the remote operated adjustable spine device 10 are shown.

The discrete displacement and control feature are shown by way of the comb 70 on the rod 30 with the comb teeth 61 on the magnet housing 60. The mating comb teeth feature interface maintains the location, carries the axial load along the control rod 30, and defines the displacement resolution.

The flat on the control tulip 90 which keeps the control to align within the control tulip also serves to keep the comb teeth 61 on the magnet housing 60 aligned with the combed teeth 70 of the rod 30. The magnet 40 is contained within the magnet housing 60. The spring 50 applies the orthogonal force to lock the device once the external magnetic field has been removed. The spring 50 is located in the annulus volume defined by the control cap 80 and the magnet housing 60. The spring is constrained in its direction of compression by the control cap 80 and the flange 62, a feature of the magnet housing 60.

### BRIEF DESCRIPTION OF OPERATION

During normal operation of this invention, the surgeon would a typical multi-axial pedicle screw 20 and a control tulip 90 with multi-axial pedicle screw across the spine section to be fused/stabilized. With these two pedicle screws installed into the pedicle bridge of the vertebrae, the control rod 30 is placed into the tulips across the gap between the two pedicle screws. The gap at this time is most likely being control by the surgeon via a clamp.

The control rod 30 is constrained from rotating on its main axis by a pair of opposing flats 201 on the control rod 30. The flats 201 align with corresponding flats within the control tulip 90. The control rod 30 is then clamped in place by securing the nut of the typical pedicle screw 20 and the control cap 80 into the control tulip 90. This is typical to the installation of a non-adjustable spine fixation set of pedicle screws and rod. A day or two after the surgery, when the patient can stand, the adjustable spine device 10 can be fine-tuned to set the proper lordosis angle. With the patient in a standing position, and properly supported, a magnetic lock release is placed against the patient's back to withdraw the magnet 40 and the comb teeth 61 to unlock the adjustable spine device 10.

The unlocking process is completed by the external magnetic device reacting with the internal device magnet 40, pulling the magnet 40 against the spring 50. This raises the magnet housing 60, thus disengaging the magnet housing's comb teeth 61 from the control rod 30 comb teeth 70. This disengagement allows the control rod to be shifted within the control tulip 90. The control rod 30 is moved along its main axis by the action of the patient, controlled by the doctor, to place the spine in the proper position, thus creating the optimum lordosis angle in the lumbar spine section. When the desired position is achieved, the external magnetic device is removed from the proximity of the patient's back, thus relocking the adjustable spine device 10. The linear holding force that the control rod 30 can withstand is a function of the shear area between the comb teeth 61 of the magnet housing 60 and the comb teeth 70 of the control rod 30. The force required to unlock the adjustable spine device is a function of the spring 50 force plus the friction force between the comb teeth. The friction force can approach zero force by positioning the posture of the patient.

The rod cover 110 is a variation on the invention that allows as an option to include a cover 110 over the comb teeth 70 of the control rod 30.

Thus, specific embodiments of a remote operated adjustable spine device have been disclosed. It should be apparent, however, to those skilled in the art that many more modifications besides those described are possible without departing from the inventive concepts herein. The inventive subject matter, therefore, is not to be restricted except in the spirit of the appended claims.

### SEQUENCE LISTING

Not Applicable.

1. A remote operated adjustable spine device comprising:
a plurality of pedicle screws;
said plurality of pedicle screws being connected with an adjustable control rod;
said control rod having at least one spline:
at least one of said two pedicle screws having at least one engaging tooth;
said at least one engaging tooth being normally maintained in said at least one spline thereby preventing movement of said at least one tooth in said at least one spline, and
wherein introduction of a magnet field disengages said at least one tooth from said at least one spline.

2. The remote operated adjustable spine device according to claim 1 wherein said plurality of pedicle screws are inserted into a spine.

3. The remote operated adjustable spine device according to claim 2 wherein a patient stands, and said magnetic field disengage said at least one tooth from said at least one spline to allow said control rod to freely move in at least one of said plurality of pedicle screws.

4. The remote operated adjustable spine device according to claim 2 wherein said plurality of pedicle screws are inserted across multiple vertebrae of said patient.

5. The remote operated adjustable spine device according to claim 4 wherein said span across multiple vertebrae corrects spine curvature.

6. The remote operated adjustable spine device according to claim 5 wherein said span allows a spine device to be inserted and held in place.

7. The remote operated adjustable spine device according to claim 1 wherein said control rod is fixedly secured to one pedicle screw.

8. The remote operated adjustable spine device according to claim 1 wherein said control rod has a stop that prevents said control rod from being displaced off of at least one pedicle screw.

9. The remote operated adjustable spine device according to claim 1 wherein said at least one spline is on a top surface of said control rod.

10. The remote operated adjustable spine device according to claim 1 wherein said at least two splines exist on opposing sides of said control rod.

11. The remote operated adjustable spine device according to claim 2 wherein two sets of remote operated adjustable spine devices are placed on mirrored sides of said spine.

12. The remote operated adjustable spine device according to claim 1 wherein said at least one of said two pedicle screws has an internal magnet.

13. The remote operated adjustable spine device according to claim 1 wherein said at least one of said two pedicle screws has an internal spring.

14. The remote operated adjustable spine device according to claim 13 wherein said internal spring forces said at least one engaging tooth into said at least one spline.

15. The remote operated adjustable spine device according to claim 1 wherein said control rod has at least one flat.

16. The remote operated adjustable spine device according to claim 15 wherein at least one of said pedicle screws has an internal cavity with at least one flat.

17. The remote operated adjustable spine device according to claim 16 wherein said flat on said control rod engages onto said at least one flat on said internal cavity of said pedicle screw prevents rotation of said pedicle screw on said control rod.

18. The remote operated adjustable spine device according to claim 1 wherein said spline is essentially square teeth or saw tooth shaped.

19. The remote operated adjustable spine device according to claim 1 wherein said person stands or is suspended to reduce spinal compression and said magnetic for is introduced to said at least one of said two pedicle screws to disengage said at least one tooth whereby said control rod moves in said pedicle screw, said magnetic field is removed to establish a displacement between said two pedicle screws.

20. The remote operated adjustable spine device according to claim 1 wherein said two pedicle screws are installed into the pedicle bridge of said vertebrae spine.

21. The remote operated adjustable spine device according to claim 1 wherein a cover shields the spline.
